(19) United States
(12) Patent Application Publication
(10) Pub. No.: US 2011/0066124 A1
(43) Pub. Date: Mar. 17, 2011

(54) Multilayer Medical Sponge

(76) Inventor: Weiru Shao, Auburndale, MA (US)

(21) Appl. No.: 12/949,761

(22) Filed: Nov. 18, 2010

Related U.S. Application Data
(60) Provisional application No. 61/394,320, filed on Oct. 18, 2010.

(51) Int. Cl. A61F 13/15

(52) U.S. Cl. 604/358

(57) ABSTRACT

A multilayer medical sponge resists damage from medical tools such as spinning drill bits and medical lasers while remaining compliant and producible in small sizes. The multilayer medical sponge includes an absorbent layer and a resistant layer. The resistant layer may include a drill-resistant material, a laser-reflective material, both types of materials, or a single material that provides both drill-resistant and laser-reflective characteristics.

### CROSS-REFERENCES TO RELATED APPLICATIONS

This application claims the benefit of U.S. Provisional Application No. 61/394,320 filed Oct. 8, 2010.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

REFERENCE TO A “SEQUENCE LISTING,” A TABLE, OR A COMPUTER PROGRAM LISTING APPENDIX

Not Applicable.

### BACKGROUND OF THE INVENTION

1. Field of the Invention

This invention relates generally to medical sponges and more particularly to medical sponges that are constructed with multiple layers.

2. Description of Related Art

Many medical procedures, including surgeries, are performed with the aid of medical lasers or drills. Frequently, laser ablation or drilling is conducted in close proximity to critical soft tissues, such as brain, blood vessels, and nerves. Working space in these surgeries is sometimes limited and may lead to accidental injuries. For example, a laser aimed at a tumor may misfire and ablate healthy tissue adjacent to the tumor. Also, a spinning drill bit removing bone may nick adjacent critical soft tissue, causing a potentially serious injury.

Techniques have been developed to protect against these hazards. These include placing medical sponges on top of critical structures. Sponges not only help to protect critical tissues, but they also help to maintain hydration during long operations.

The inventor has recognized that conventional medical sponges sometimes fall short in performing their protective functions. For example, laser light may penetrate sponges and damage underlying tissues. Sometimes sponges can ignite when struck by laser light. Intraoperative fires may be particularly damaging in procedures for which oxygen is fed to the operative field, such as endoscopic laryngeal, bronchial, and tracheal surgeries.

Spinning drill bits also present a hazard. As is known, drill bits used in medical procedures may be composed of fluted metal or other materials, and may have roughened or abrasive surfaces. Some include abrasive particles such as diamond bur. All of these various drilling and grinding tools are collectively referred to herein as “drill bits.” If a spinning drill bit accidentally touches a sponge, it may “catch” on the sponge, causing the sponge to become dislodged from its protective position and exposing the underlying tissues to damage. In some cases, the sponge may be caused to wrap around the drill bit and form a fast spinning ball of material. The size and speed of the spinning material may be enough to damage adjacent tissues.

### BRIEF SUMMARY OF THE INVENTION

The inventor has recognized and appreciated that the safety of surgeries and other medical procedures may be improved by a multilayer medical sponge. In accordance with one embodiment, the multilayer medical sponge includes at least two layers, an absorbent layer and a resistant layer. The absorbent layer may be placed against sensitive tissues to cover them and provide hydration during medical procedures, whereas the resistant layer may face away from these tissues to help protect them from damage resulting from various hazards, such as impingement of medical tools, including spinning drill bits or laser light, upon the multilayer sponge.

In accordance with another embodiment, a multilayer sponge includes an absorbent layer and a resistant layer adjacent to the absorbent layer. Each layer has a surface facing outward. The outward-facing surface of the resistant layer is smoother than that of the absorbent layer.

In accordance with yet another embodiment, a medical sponge includes a first layer and a second layer. The first layer includes an absorbent material. The second layer is affixed to the first layer and includes at least one of a drill-resistant material and a laser-reflective material.

In accordance with still another embodiment, a method of making a multilayered medical sponge includes the acts of providing an absorbent material and providing a slippery material. The method further includes the act of affixing together the absorbent material and the slippery material to form a multilayered structure.

In accordance with a further embodiment, a medical sponge includes a first layer constructed from a biocompatible material and a second layer adhered to the first layer. The second layer is constructed from deflection material.

In accordance with a still further embodiment, a method of preventing damage with a medical sponge during a medical operation includes providing a medical sponge having a first layer constructed from a biocompatible material adhered to a second layer constructed from a deflection material. The method further includes placing the medical sponge proximate to at least one critical structure of a medical patient and using a medical tool proximate to the at least one critical structure, wherein the second layer of the medical sponge is positioned to substantially deflect the medical tool.

### BRIEF DESCRIPTION OF THE SEVERAL VIEWS OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a perspective view of an exemplary multilayer medical sponge;

FIG. 2 is a side view of the exemplary multilayer medical sponge of FIG. 1;

FIG. 3 is a simplified view of an exemplary medical procedure wherein the multilayer medical sponge of FIGS. 1-2 may be used;

FIG. 4 is a side view of another exemplary multilayer medical sponge;

FIG. 5 is a simplified view of another exemplary medical procedure wherein the multilayer medical sponge of FIG. 4 may be used;

FIG. 6 is a side view of yet another exemplary multilayer medical sponge; and

FIG. 7 is a flowchart showing an exemplary process for making multilayer medical sponges.

### DETAILED DESCRIPTION OF THE INVENTION

Techniques, including a preferred embodiment of the invention, are described hereinbelow for providing a multilayer medical sponge to help protect medical patients against injuries arising from various hazards such as contact with spinning drill bits and other tools or instruments and/or from impingement with laser light. These techniques may be employed to make a multilayer medical sponge and to use a multilayer medical sponge to improve the safety of medical procedures.

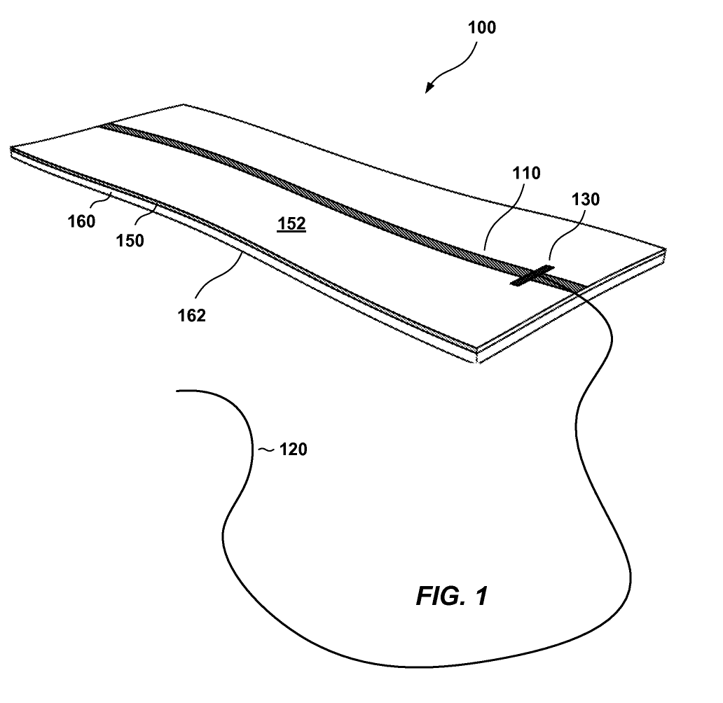

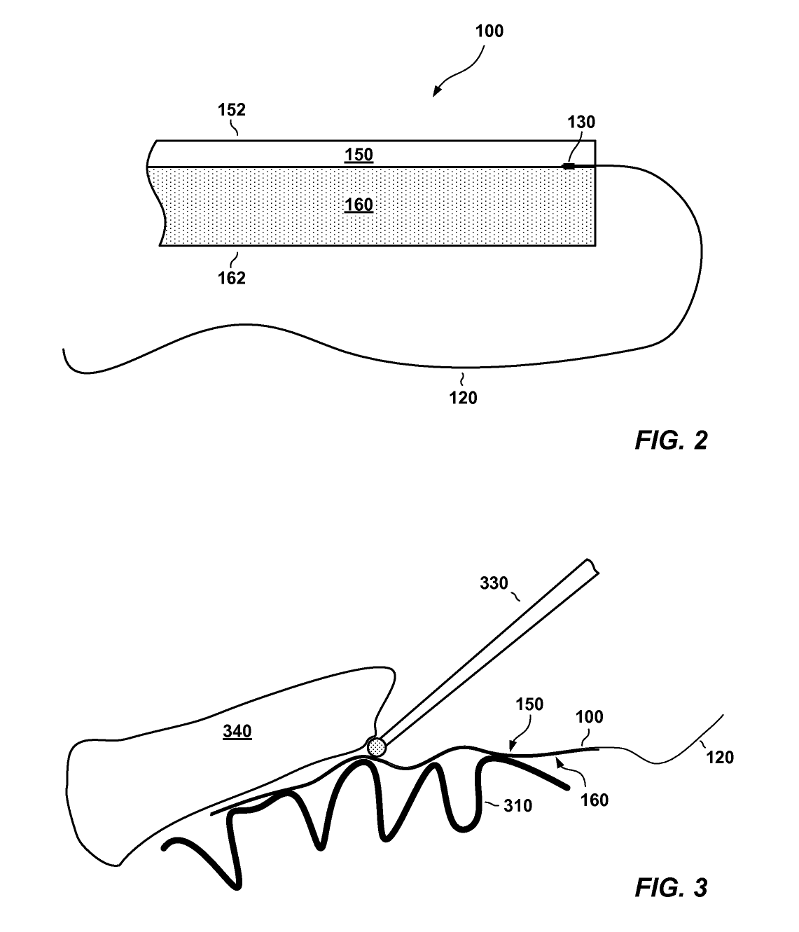

FIGS. 1 and 2 show an exemplary multilayer medical sponge 100 which provides drill-resistant characteristics. The sponge 100 includes a first layer 160 and a second layer 150. The first layer 160 is made of an absorbent material and has an outward-facing surface 162. The second layer 150 is made of a resistant material and has an outward-facing surface 152. Optionally, a radiopaque marker 110 is applied to the sponge 100 and a string 120 is attached to the sponge using, for example, stitching 130. The radiopaque marker 110 may be imbedded or painted within sponge 100, e.g., between the layers 160 and 150, for use in localizing and identifying the sponge 100 within a surgical field using x-rays. The string 120 may be applied in any suitable way, such as between the layers 160 and 150 or to the top or bottom of the sponge 100. The string 120 may assist in retrieving or relocating the sponge 100 during a procedure.

The layers 160 and 150 are affixed together; for example, they may be stitched together. Alternatively, they may be affixed by gluing, heating, melting, bonding, or ultrasonic welding, or by using any other suitable technique. The resistant layer 150 may be also applied in a liquid or semi-liquid state, such as by rolling or spraying, and then allowed to dry or cure.

The absorbent layer 160 may be made from a soft, biocompatible, absorbent material that is safe when placed in contact with sensitive tissues and is capable of retaining water for maintaining hydration of those tissues. Non-limiting examples include cotton, gauze, rayon, silk, or polyester. Other materials may be used, and such materials are generally fibrous, porous, or both, to promote wicking action and absorption and retention of bodily fluids.

The resistant layer 150 provides drill-resistant characteristics. In this manner, the resistant layer 150 may be smoother and substantially slipperier than the absorbent layer 160. For example, the surface 152 may have a lower coefficient of friction with metal or diamond bur than the surface 162. The increased slipperiness of the resistant layer 150 may help to prevent the sponge 100 from being dislodged by frictional contact with medical tools and instruments, such as spinning drill bits. The outer surface 152 of the resistant layer may be substantially free of loose fibers, gaps, or other structures that are prone to cause entanglement with a spinning drill bit.

The resistant layer 150 may also be semi-rigid. In this context, “semi-rigid” means relatively flexible for radii of curvature above a certain level, but relatively stiff for radii of curvature below this level. In one example, the transition between stiffness and flexibility is a radius of about 5 mm. Drill bits used for medical purposes typically have diameters in the range between 0.5 mm and 8 mm. Because the resistant layer 150 causes the sponge 100 to be stiff on the scale of typical drill bits, the resistant layer helps to prevent the sponge 100 from deforming easily around the drill bits and thereby being drawn around them to form spinning balls of material. At the same time, the semi-rigidity of the resistant layer 150 allows the sponge 100 to remain pliable and compliant on larger scales. Therefore, the sponge 100 may still be draped over sensitive tissues and caused to readily conform to their shapes.

The degree of rigidity of the resistant layer 150 may be varied based on the intended use, and different sponges 100 may be provided having different degrees of rigidity. For example, when using a sponge 100 in the vicinity of small drill bits, e.g., in the 0.8 mm range, a sponge 100 may be selected that has a resistant layer 150 composed and sized so as to place the transition between stiffness and flexibility at a smaller radius, such as 2 mm. Conversely, when using a sponge 100 in the vicinity of large drill bits, e.g., in the 8 mm range, a different sponge 100 may be selected that has a resistant layer 150 composed and sized so as to place the transition between stiffness and flexibility at a larger radius, such as 1 cm. An assortment of sponges 100 having different transition radii may be provided.

The resistant layer 150 may also be tough. By “tough” it is meant that the resistant layer 150 may not be easily worn away by brief contact with spinning drill bits. Once the resistant layer 150 has been partly or completely worn away by repeated contact with a drill bit, medical personnel may replace the sponge 100 with a new one.

To provide drill-resistance, the resistant layer 150 may be composed of materials having one or more of the above characteristics, i.e., smoothness, slipperiness, freedom from loose fibers or gaps, semi-rigidity, or toughness. Examples of suitable materials may include, but are not limited to, Gore-Tex®, nylon, Silastic®, Teflon®, and aluminum foil.

The thickness of the resistant layer 150 depends upon the materials used and the desired level of semi-rigidity. In one example, the resistant layer 150 is made of Teflon and has a thickness of 0.06 mm. Using this material and thickness, a multilayer sponge 100 may be constructed having an overall dry thickness of 0.8 mm, which is approximately the same as the thickness of a conventional medical sponge. Therefore, the sponge 100 provides protective functions that are absent from conventional sponges without requiring much if any additional size or bulkiness. The fact that the sponge 100 may be made to be no larger than conventional sponges confers a great advantage in surgeries conducted in very small spaces.

When used in a surgical procedure, the multilayer sponge 100 may be inserted into a surgical field and placed against sensitive tissues with the absorbent layer 160 contacting the sensitive tissues, thereby allowing the absorbent layer 160 to cover and hydrate them. The resistant layer 150 of the multilayer sponge 100 may face outwardly, away from the sensitive tissues. The resistant layer 150 may thereby form a protective shield over the sensitive tissues to help guard them against intraoperative injury.

FIG. 3 shows an example of a surgical procedure in which a multilayer sponge 100 may be employed to help to protect against damage from a surgical tool, such as a spinning drill bit 330. The sponge 100 may be inserted into a surgical field and placed directly over sensitive tissue, such as brain tissue 310. The sponge 100 may be placed with its absorbent layer 160 facing toward the brain tissue 310 and its resistant layer 150 facing away from the brain tissue 310. The surgical procedure may require that certain portions of bone, such as skull bone 340, located close to the brain tissue 310, be removed using the drill bit 330. With conventional sponges, this procedure may pose a risk that inadvertent contact between the spinning drill bit 330 and a conventional sponge would dislodge the sponge from its protective position and possibly cause the sponge to wrap around the drill bit 330 and form a dangerously spinning ball of material. However, with the multilayer sponge 100, this risk is significantly reduced. The tough, slippery, and/or semi-rigid qualities of the resistant layer 150 of the multilayer sponge 100 enable it simply to yield to contact with the spinning drill bit 330 without becoming dislodged. Typically, the frictional forces between the absorbent layer 150 and the underlying tissue 310 are relatively high, especially when the absorbent layer 150 is wet, compared to the frictional forces between the drill bit 330 and the resistant layer 150. Consequently, the sponge 100 tends to remain in place. Also, the semi-rigid quality of the resistant layer 150 typically prevents the sponge 100 from deforming around the spinning drill bit 330 and becoming entangled. Contact between the spinning drill bit 330 and the sponge 100 may cause localized heating within the sponge 100. However, the degree of heating is much less than with conventional sponges due to the slipperiness of the resistant layer 150. Also, since the resistant layer 150 is adjacent to and generally in direct contact with the wet absorbent layer 160, heat is quickly drawn away from the point of contact and dispersed, posing little risk of harm. If a medical procedure results in repeated or frequent contact between the drill bit 330 and the sponge 100, medical personnel may be well advised to replace the sponge 100, or at least to inspect the resistant layer 150 for signs of wear before exposing the sponge 100 to additional contact with the drill bit. It is understood, therefore, that the drill-resistant quality of the resistant layer 150 is not intended to be absolute but rather relative and significantly improved as compared with that of conventional sponges.

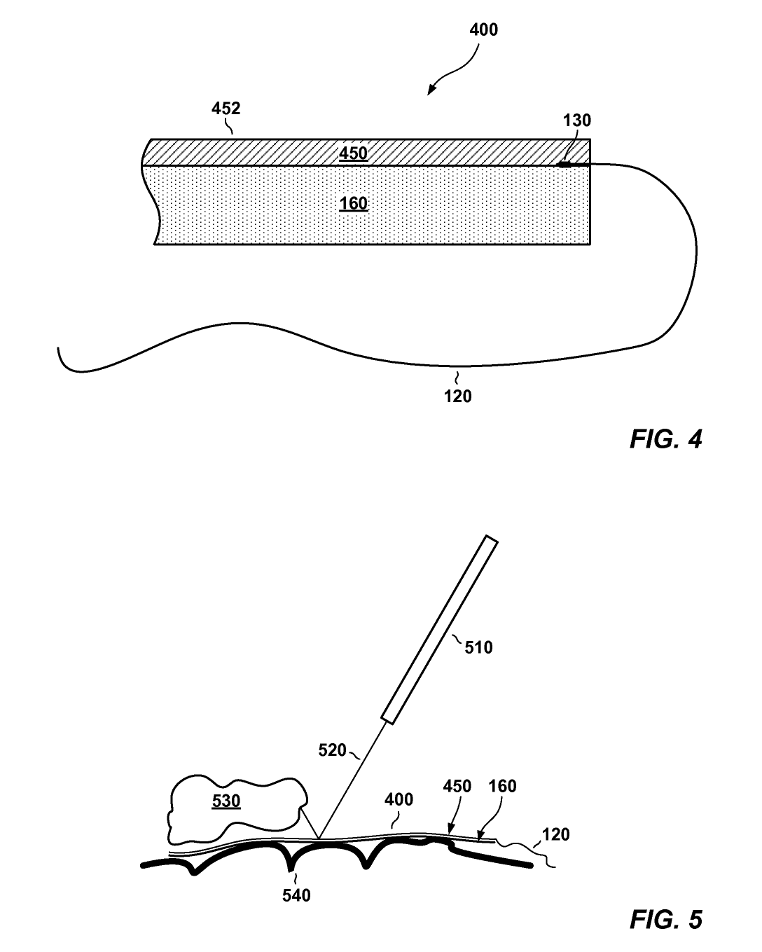

FIG. 4 shows another illustrative example 400 of a multilayer medical sponge. The multilayer sponge 400 provides laser-reflective characteristics. The medical sponge 400 may be constructed of many of the same materials as the medical sponge 100. For instance, the medical sponge 400 may include an absorbent layer 160, a string 120, stitching 130, and a radiopaque marker 110, which are all similar to those of the sponge 100. However, in place of the resistant layer 150, which provides drill-resistant characteristics, the multilayer medical sponge 400 includes a resistant layer 450 having laser-reflective characteristics. The laser-reflective, resistant layer 450 has a surface 452 which is reflective of laser light, such as light emitted from Argon, KTP, or CO₂ lasers. The resistant layer 450 may include any suitable material that is at least partially reflective of laser light, such as metal, or that has a surface 452 that is at least partially reflective of laser light. In one example, the resistant layer 450 is composed of metal foil, such as aluminum foil. Other metal foils or materials may be used. The resistant layer 450 may be flame retardant or flame resistant to prevent interoperative fires.

The resistant layer 450 may be composed of a material that is partially laser-absorptive. For instance, aluminum foil may both reflect and absorb laser energy. A partially reflective, partially absorptive resistant layer 450 may provide advantages over a purely reflective layer during medical procedures, because any stray reflections from the resistant layer 450 will have reduced energy as compared with incident laser light, and thus may present less risk to surrounding tissues, materials, and medical personnel. Absorption of laser energy by the partially absorptive layer 450 may cause localized heating of the resistant layer 450. However, it should be noted that this layer is adjacent to the absorbent layer 160, which is generally wet and provides some degree of heat sinking Therefore, any heat absorbed as a result of brief laser pulses may be quickly conducted away from the point of contact and harmlessly dispersed into the wet absorbent layer 160 and surrounding tissues. Heat conduction and cooling are enhanced through the use of metal in the resistant layer 450, owing to the high heat conductivity of metal.

The layers 160 and 450 may be affixed together by any suitable method. In one example, they may be stitched together. Alternatively, for instance, they may be affixed by gluing, heating, melting, bonding, or ultrasonic welding. The resistant layer 450 may also be applied in a liquid or semi-liquid state, such as by rolling or spraying, and then allowed to dry or cure. It may be applied in a gaseous state by evaporation directly onto the absorbent layer 160. The resistant layer 450 can be made to be very thin so that it adds little additional bulk to the sponge 400 as compared with conventional sponges and substantially retains the pliability of conventional sponges.

FIG. 5 shows an example of a surgical procedure in which a multilayer sponge 400 may be employed to help to protect against damage from laser light. Here, a medical laser 510 is used to ablate undesired tissue 530, such as a tumor or scar, which is located adjacent to critical soft tissue 540, such as the brain, a nerve, or a blood vessel. In preparing the surgical field, a multilayer sponge 400 may be placed over the critical tissue 540 and between the critical tissue 540 and the undesired tissue 530. The sponge 400 is placed with its absorbent layer 160 facing toward the critical tissue 540 and its resistant layer 450 facing away from the critical tissue 540. The laser 510 is generally aimed at the undesired tissue 530 and made to emit bursts of laser light 520. Sometimes, it is possible for misalignments to occur or for the laser 510 to misfire, causing the laser light 520 to strike the sponge 400 rather than the undesired tissue 530. Also, it is sometimes possible for the laser light 520 to completely penetrate the tissue 530 and strike the sponge 400 beneath the tissue 530. With conventional sponges, these scenarios present a risk that laser light 520 may pass directly through the sponge and damage the underlying critical tissue 540. There is also a risk that the sponge could ignite, particularly when oxygen is fed to the operative field, as is done during certain endoscopic laryngeal, bronchial, and tracheal surgeries. These risks are significantly reduced, however, when using a multilayer sponge 100 equipped with a laser-reflective, resistant layer 450. Rather than penetrating the sponge 400, the incident laser light 520 may reflect off of the sponge 400 and away from the underlying critical tissue 540, thereby preventing harm to the underlying critical tissue 540. The resistant layer 450 of the sponge 400 may be intentionally made to be only partially reflective, in which case some of the energy of the laser light 520 may be absorbed. Although localized heating may occur at the point of contact, heat generally spreads out rapidly, particularly when the resistant layer 450 is metallic. The heat may then be harmlessly dispersed by the wet absorbent layer 160 and surrounding tissues.

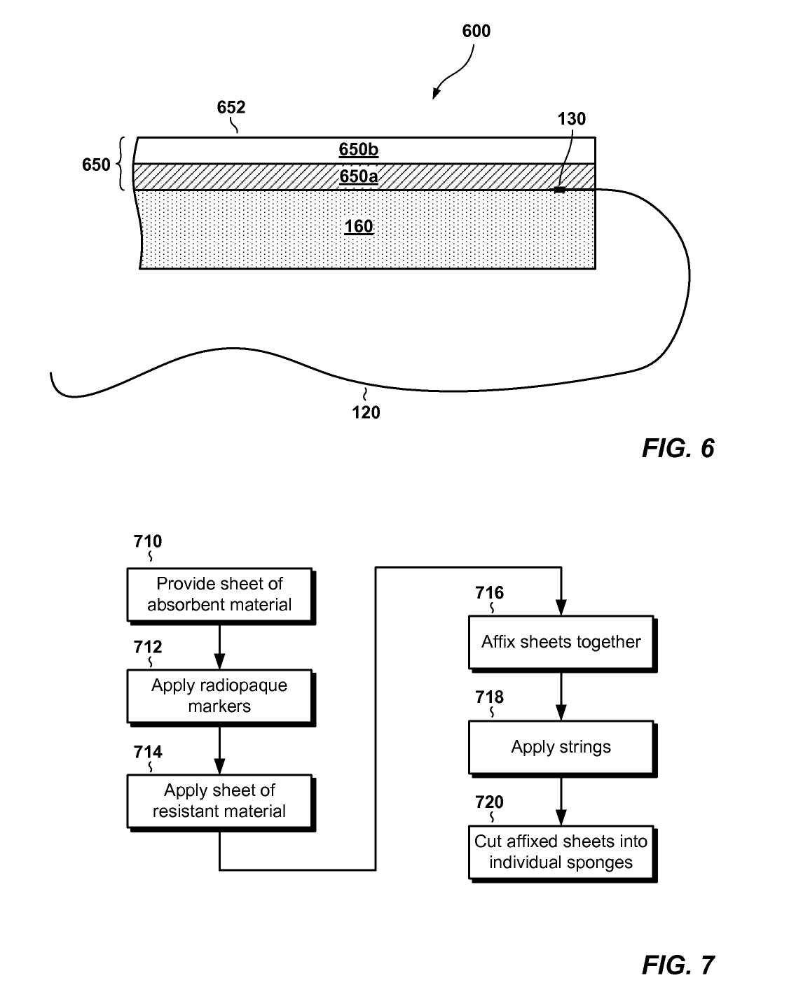

FIG. 6 shows yet another illustrative example 600 of a multilayer medical sponge. Like the sponges 100 and 400, the multilayer sponge 600 includes an absorbent layer 160 and a resistant layer. The absorbent layer 160 is similar to that described in connection with the sponges 100 and 400 above. However, the resistant layer 650 of the sponge 600 includes sublayers 650a and 650b. One of the sublayers may be drill-resistant, like the layer 150, and the other may be laser-reflective, like the layer 450. For example, the sublayer 650a may be laser-reflective and the sublayer 650b may be drill-resistant. Each sublayer 650a and 650b may be composed and arranged substantially as described above for the respective layers 450 and 150. The laser-reflective sublayer 650a reflects incident laser light, and the drill-resistant sublayer 650b protects the sponge 600 against contact with spinning drill bits and other medical tools and instruments. The multilayer sponge 600 is thus well suited for medical procedures involving the user of both lasers and drills.

Because the laser-reflective sublayer 650a is positioned beneath the drill-resistant sublayer 650b, incoming laser light must pass through the drill-resistant sublayer 650b before striking the laser-reflective sublayer 650a and again after being reflected by the laser-reflective sublayer 650a. To avoid ablating the drill-resistant sublayer 650b, the drill-resistant sublayer 650b may be composed of a material that is substantially transparent to laser light. As is known, different lasers emit laser light at different wavelengths. In one example, a material is provided for the drill-resistant layer 650 that is transparent to laser light from most or all lasers currently used in medical procedures. In another example, different materials are provided that are transparent to different bands of laser light. For instance, a selection of different types of sponges 600 may be provided, wherein each sponge in the selection has a drill-resistant layer 650b that is transparent to a particular band of laser light. The bands may include, for example, a visible band, an infrared band, and an ultraviolet band. A sponge 600 may then be chosen from the selection based on matching its transparent band to the known emission wavelength of the particular laser to be used. For example, a sponge 600 with a drill resistant layer 650b that is transparent to visible light may be chosen for use in connection with a laser known to emit in the visible spectrum. A particular example may be to provide a drill-resistant layer 650b made from clear silicon for use with Argon medical lasers emitting in the visible spectrum.

The layers, including the sub-layers, of the multilayer sponge 600 may be affixed together in any suitable way. These include, for example, the ways described above for affixing the layers 150, 160, and 450 of the sponges 100 and 400. In addition, the sublayers 650a and 650b may be made very thin so that they add little additional bulk to the sponge 600 and the sponge 600 may have a similar thickness to that of conventional sponges.

The materials used to construct the multilayer sponges 100, 400, and 600 are preferably non-toxic and free of any substances that may cause negative tissue reactions. When the sponges 100, 400, and 600 are used in the proximity of spinning drill bits or laser light, it is expected that a small volume of fine debris may be ejected from the sponges. Some of this fine debris may remain in the patient's body after surgery. Therefore, it is important to the long-term health of patients that the materials of the sponges elicit no ill effects, either in the short term or in the long term.

FIG. 7 shows an exemplary process for making multilayer sponges. This process may be applied in making any of the multilayer sponges 100, 400, or 600. Preferably, many sponges are made at a time. At step 710, an absorbent material is provided. This material is used to form the absorbent layer 160 of each of the sponges to be made, and may include, for example, cotton, gauze, rayon, silk, and polyester. The absorbent material may be provided in the form of a sheet and fed from a roll. At step 712, radiopaque markers 110 may be applied to the sheet of absorbent material. These may be rolled, painted, printed, or adhered to the absorbent material, for example. At step 714, resistant material may be applied over the absorbent material. The resistant material may be applied in the form of a sheet fed from a roll and laid over the sheet of absorbent material. To make the multilayer sponges 100, the resistant material may be composed of a drill-resistant material, such as Gore-Tex®, nylon, Silastic®, Teflon®, or aluminum foil, for example. To make the multilayer sponges 400, the resistant layer may be composed of a laser-reflective material, such as metal, including metal foil, or other reflective materials. To make the multilayer sponges 600, two distinct sublayers may be applied. A laser-reflective material may be applied first to form the sublayer 650a, and a drill-resistant material may be applied second to form the sublayer 650b. At step 716, the sheet of absorbent material and the sheet (or sheets) of resistant material may be affixed together. The sheets may be affixed using any suitable technique, including but not limited to stitching, gluing, heating, melting, bonding, and ultrasonic welding. At step 718, strings 120 may be attached, using stitching 130 or other means. At step 720, the multilayer structure may be cut into individual sponges.

It is understood that the sequence of steps shown in FIG. 7 may be varied. The specific sequence shown is provided merely as an example and should not be regarded as limiting. Acts may be performed in a different order, and some acts may be performed simultaneously. For instance, strings and radiopaque markers can be applied at any point in the process. Although the process begins by providing a sheet of absorbent material, upon which other materials are applied, it could alternatively begin with a sheet of resistant material. Other materials could then be applied to the resistant material.

The multilayer sponges as described herein help to protect patients against injuries that can occur during medical procedures from inadvertent effects of medical tools, such as spinning drill bits and lasers. The sponges perform their protective function while continuing to fulfill their conventional role of covering and hydrating sensitive tissues.

Preferably, the multilayer medical sponges 100, 400, and 600 are provided in a range of sizes and shapes, as desired for different medical uses. Typically, sponges are rectangular and range in size from 0.5 cm by 1 cm to 2 cm by 10 cm, although these are merely examples. Sponges may be provided in any suitable size and shape.

Having described certain embodiments, numerous other embodiments or variations can be made. For example, as shown and described, the resistant layers of the multilayer sponges 100 and 400 may be either drill-resistant or laser-reflective. However, a resistant layer may also be both drill-resistant and laser-reflective. For example, certain materials, such as aluminum foil and certain plastics or other materials, may provide both drill-resistance and laser-reflectiveness. The same resistant layer may therefore be used to protect against both drill bits and lasers.

Also, the resistant layers of the various multilayer sponges may be resistant to other potentially harmful agents besides drill bits and laser light. For example, they may protect against contact with medical tools such as scalpels and probes. In addition, the resistant layers of the various sponges may help to protect or shield against debris such as bone chips produced by saws, drills, or other equipment. The same characteristics that allow the multilayer sponges to be drill-resistant may also provide protection against other potentially harmful agents.

One may surmise from the foregoing that the surface 452 of the laser-reflective layer 450 is flat and smooth. However, this is merely an example. Alternatively, the surface 452 may be provided with a surface texture for the purpose of diffusing incident laser light. According to one variant, the texture may include a predetermined pattern of bumps and/or ridges. According to another variant, the laser-reflective material that forms the layer 450 may be rolled, painted, sprayed, or evaporated onto the absorbent layer 160. The resulting film of laser-reflective material may then conform to the naturally fibrous and/or porous surface of the absorbent layer 160. Surface irregularities may then afford the resistant layer 450 with a laser-diffusive capability. Laser light that hits the resistant layer 450 may be reflected in a diffuse pattern over a large area. Such diffusion of laser light may have positive safety effects because diffused laser light striking any object will have very little power compared to a concentrated beam and therefore may pose less risk of harm.

As shown and described, the layers and sublayers of each of the multilayer sponges 100, 400, and 600 are aligned with other layers of the respective sponges and have the same perimeters and areas. This is merely an example. Alternatively, one layer may be made to be intentionally larger or smaller than another layer. For instance, the resistant layers 150/450/650 may be made to overhang the edges of the absorbent layers 160 to help protect the edges of the absorbent layers from drills, laser light, and/or other potentially harmful agents. Also, the absorbent layers 160 may be made to extend beyond the boundaries of the respective resistant layers 150/450/650 to allow for increased fluid absorption or other benefits. Similarly, either of the sublayers 650a and 650a may be made larger or smaller than the other based on desired performance.

One may surmise that the resistant layers 150/450/650 are non-porous and impermeable. Although this may be the case in some instances, it is not required. For example, the resistant layers may be composed of porous materials, or they may be perforated to allow them to “breathe” and exchange gases with their surroundings. Any pores or perforations in drill-resistant layers may be much smaller than the diameters of the drill bits with which the sponges are used, to prevent the drill bits from “catching” on the pores or perforations.

Although the multilayer sponges shown and described have two layers, or three layers, if the two sublayers 650a and 650b are to be considered as separate layers, this is merely an example. Additional layers may be provided.

Also, although the sponges 100, 400, and 600 are described in connection with certain surgeries, they may be used in any medical procedure that employs sponges.

The resistant layers 150, 450, and 650 may be regarded as “deflection materials.” These are materials that deflect, resist, repel, or otherwise prevent damage caused by a medical tool. The materials identified above for constituting the resistant layers 150, 450, and 650 are also regarded as deflection materials.

Also, it is understood that the layers of material need not be made from solid sheets. For example, the material used to form the resistant layer may be sprayed, rolled, painted, powder coated, or evaporated onto an absorbent layer, or it may be applied in some other way. Also, the resistant layer may be provided as a sticky liquid sheet. The absorbent material may then be applied to the resistant layer as a solid sheet or blown on as loose particles that adhere to the resistant layer.

Further, it is not strictly required that the different layers or sublayers be formed from different materials. For example, they may be formed from a single material that is treated differently on its opposing surfaces. For instance, a material such as polyester may be provided in the form of a fibrous sheet. During fabrication, one side of the sheet could be kept cool while the other side is heated. The heated side could be allowed to melt to form a smooth, slippery surface, while the cool side is maintained in a cool state to remain fibrous and absorbent.

Although certain embodiments are disclosed herein, it is understood that these are provided by way of example only and the invention is not limited to these particular embodiments. Those skilled in the art will therefore understand that various changes in form and detail may be made to the embodiments disclosed herein without departing from the scope of the invention.

Various aspects of the present invention may be used alone, in combination, or in a variety of arrangements not specifically discussed in the embodiments described in the foregoing. The invention is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A multilayer medical sponge, comprising:
an absorbent layer having a surface, the surface facing outward; and
a resistant layer, adjacent to the absorbent layer and having a surface, the surface facing outward and being smoother than the surface of the absorbent layer.

2. The multilayer medical sponge as recited in claim 1, wherein the resistant layer is semi-rigid.

3. The multilayer medical sponge as recited in claim 2, wherein the surface of the resistant layer is slipperier than the surface of the absorbent layer.

4. The multilayer medical sponge as recited in claim 1, wherein the resistant layer is free of visibly loose fibers and pores.

5. The multilayer medical sponge as recited in claim 1, wherein the resistant layer comprises at least one of Gore-Tex, Nylon, Silastic, Teflon, and aluminum foil.

6. The multilayer medical sponge as recited in claim 1, wherein the resistant layer comprises a laser-reflective material.

7. The multilayer medical sponge as recited in claim 6, wherein the laser-reflective material is substantially reflective of light emitted from at least one of Argon, KTP, and CO₂ lasers.

8. The multilayer medical sponge as recited in claim 6, wherein the laser-reflective material is substantially flame retardant.

9. The multilayer medical sponge as recited in claim 1, wherein the resistant layer comprises a first sublayer and a second sublayer, the first sublayer including a material that is reflective of laser light and the second sublayer including a material that is slipperier than the absorbent layer.

10. The multilayer medical sponge as recited in claim 9, wherein the first sublayer is immediately adjacent to the absorbent layer.

11. The multilayer medical sponge as recited in claim 10, wherein the second sublayer is substantially transparent.

12. A medical sponge, comprising:
a first layer including an absorbent material; and
a second layer, affixed to the first layer and including at least one of a drill-resistant material and a laser-reflective material.

13. The medical sponge as recited in claim 12, wherein the second layer is semi-rigid.

14. The medical sponge as recited in claim 12, wherein the second layer is slipperier than the absorbent layer.

15. The medical sponge as recited in claim 12, wherein the second layer comprises a first sublayer and a second sublayer, the first sublayer including the laser-reflective material and the second sublayer including the drill-resistant material.

16. The medical sponge as recited in claim 15, wherein the laser-reflective material comprises metal and the drill-resistant material is substantially transparent.

17. A method of making a multilayered medical sponge, comprising:
providing an absorbent material;
providing a slippery material; and
affixing together the absorbent material and the slippery material to form a multilayered structure.

18. The method as recited in claim 17, wherein the acts of providing comprise providing a slippery material that is substantially more resistant than the absorbent material to at least one of laser ablation and entanglement with sharp objects.

19. The method as recited in claim 17, wherein the act of affixing the absorbent material and the slippery material comprises at least one of stitching, heating, melting, gluing, bonding, rolling, painting, spraying, evaporating, and ultrasonic welding.

20. The method as recited in claim 17, further comprising cutting the multilayered structure into individual sponges.
